(19) United States
(12) Patent Application Publication
(10) Pub. No.: US 2018/0371544 A1
(43) Pub. Date: Dec. 27, 2018

(54) Sequencing Methods

(71) Applicant: Northeastern University, Boston, MA (US)

(72) Inventors: Konstantin Khrapko, Newton, MA (US); Sfia Annis, Roxbury Crossing, MA (US); Jonathan Lee Tilly, Windham, NH (US); Dori Cousins Woods, Londonderry, NH (US); Slava Epstein, Dedham, MA (US)

(21) Appl. No.: 16/066,103

(22) PCT Filed: Dec. 30, 2016
(86) PCT No.: PCT/US16/69519

§ 371 Date: Jun. 26, 2018

Related U.S. Application Data
(60) Provisional application No. 62/273,702, filed on Dec. 31, 2015.

(51) Int. Cl. C12Q 1/6876; C12Q 1/6869

(52) U.S. Cl. CPC C12Q 1/6876; C12Q 1/6869

(57) ABSTRACT

Disclosed are compositions and methods related to the use of unique molecular identifiers (UMIs) to improve the error-correction capability of third generation sequencing and similar approaches that involve high precision reading of long segments of single DNA molecules.

### RELATED APPLICATIONS

This application claims priority to U.S. Provisional Application Nos. 62/273,702, filed Dec. 31, 2015, which is hereby incorporated by reference in its entirety.

### BACKGROUND

The long read/single molecule “third generation” sequencing technologies have become mainstream in de novo sequencing as well as high fidelity resequencing of genomes. The main advantage of such sequencing technologies is the large length of the reads possible (approaching 15,000 bp). However, a common disadvantage of these technologies is a high error rate resulting mainly from “instrumental error”, i.e. misinterpretation of the signals, such as fluorescence bursts (PacBio) or current changes (NanoPore), associated with reading certain nucleotides. The result is individual reads that are long but error prone. A common solution to this problem is to generate a large number of reads and average out errors upon alignment of the reads to a consensus sequence, which works well in situations where the goal is sequencing individual genomes.

However, averaging out errors upon alignment is not a viable strategy in cases where mixtures of similar genomic or DNA sequences are being analyzed, such as is the case in complex mixtures of microbes in microbiome studies. Analysis of mixtures does not offer a common consensus between DNA sequences, so error correction via amassing the number of reads is not possible. The current technology used for error correction by PacBio is called Circular Consensus Sequence (CCS), which takes advantage of the ability of a polymerase to go around a circular template multiple times, thereby sequencing the same molecule several times. In Nanopore technology, an analogous (though much less efficient) approach is the sequential sequencing of both strands of the same double stranded DNA molecule.

While the CCS approach works well for reads of up to about 1,500 bp in length, at longer read lengths the polymerase fails to circle the double-stranded template a sufficient number of times for efficient error correction. Thus, the current technologies are limited to relatively short read lengths when applied to complex mixtures of closely related nucleic acid sequence. This can be problematic in many applications. For example, analysis of complex mixtures is key for a detailed characterization of microbiome(s), and also for the identification of closely-related bacterial strains, which may be pathogenic, drug-resistant, or otherwise altered. Finally, an additional problem is that analysis of many types of samples requires nucleic acid amplification (e.g., using PCR) prior to sequencing because the samples may contain only small amounts of nucleic acid, may be difficult to replenish and may be extremely complex. PCR amplification can be particularly problematic, because PCR derived artifacts are very difficult to distinguish from real genetic differences in single molecule sequencing analysis of complex mixtures, and there is therefore currently no efficient way to distinguish a PCR error from a low frequency sequence variant in long DNA fragments.

Thus, there is a great need for improved methods and compositions that improve the error-correction capability associated with the sequencing of long segments of single nucleic acid molecules from complex mixtures.

### SUMMARY

In certain aspects, provided herein are compositions and methods related to the use of unique molecular identifiers (UMIs) to improve the error-correction capability of third generation sequencing and similar approaches that involve high precision reading of long segments of single DNA molecules.

In certain aspects, provided herein are primer-adapter nucleic acid molecules and populations of primer-adapter nucleic acid molecules for sequencing a region of a target nucleic acid. In some embodiments, each primer-adapter nucleic acid molecule comprises, in 5′ to 3′ order: (a) a generic primer region having a nucleotide sequence shared among primer-adapter nucleic acid molecules and that is not complementary to a sequence of the target nucleic acid; (b) a unique molecular identifier (UMI) region having a sequence that differs between each member of the primer-adapter nucleic acid molecules; and (c) a gene-specific primer region having a nucleotide sequence shared among the primer-adapters nucleic acid molecules and that is complementary to the sequence located at the 3′ end of the region of the target nucleic acid to be sequenced.

In some embodiments of the primer-adapter nucleic acid molecules provided herein, the generic primer region has a sequence that is not complementary and that does not correspond to any sequence in the target nucleic acid molecule. In some embodiments, the generic primer region has a sequence that is not complementary and that does not correspond to any sequence in the genome of the organism or virus in which the target nucleic acid molecule is naturally present. In some embodiments, the generic primer region has a sequence that is not complementary and that does not correspond to any sequence in the genome of any known organism or virus. In some embodiments, the generic primer region is of between 15 and 40 nucleotides in length (e.g., of 15, 16, 17, 18, 19, 20, 21, 22, 23, 24, 25, 26, 27, 28, 29, 30, 31, 32, 33, 34, 35, 36, 37, 38, 39 or 40 nucleotides in length).

IN certain embodiments of the primer-adapter, the UMI region is a degenerate nucleotide sequence. For example, in some embodiments the UMI region is a 4-fold degenerate nucleotide sequence. In some embodiments, the UMI region is a 3-4old degenerate nucleotide sequence (e.g., consisting of A, T and C nucleotides). In some embodiments, the UMI region is between 10 and 20 nucleotides in length (e.g., 10, 11, 12, 13, 14, 15, 16, 17, 18, 19 or 20 nucleotides in length).

In some embodiments of the primer-adapter, the gene-specific primer region is of between 15 and 40 nucleotides in length (e.g., of 15, 16, 17, 18, 19, 20, 21, 22, 23, 24, 25, 26, 27, 28, 29, 30, 31, 32, 33, 34, 35, 36, 37, 38, 39 or 40 nucleotides in length). In some embodiments, the melting temperature of the gene-specific primer region for its complement is lower than the melting temperature of the generic primer region (e.g., lower by at least 1, 2, 3, 4, 5, 6, 7, 8, 9, 10, 11, 12, 13, 14, 15, 16, 17, 18, 19 or 20 ° C. in PCR buffer). In some embodiments, the gene-specific primer region comprises one or more U nucleotides (e.g., at least 1, 2, 3, 4, 5, 6, 7, 8 or 9 U nucleotides). In some embodiments, the gene-specific primer region comprises U nucleotides in place of T nucleotides (e.g., it does not include any T nucleotides).

In some embodiments, the primer-adapter nucleic acid molecules described herein also include a spacer region located immediately 3′ of the generic primer region. In some embodiments, the spacer region is of between 10 and 100 nucleotides in length (e.g., 10, 11, 12, 13, 14, 15, 16, 17, 18, 19, 20, 21, 22, 23, 24, 25, 26, 26, 28, 29, 30, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length). In some embodiments, the spacer sequence region has a sequence that does not include G nucleotides (e.g., a sequence that only includes A, T and C nucleotides).

In certain embodiments, the primer-adapter nucleic acid molecules described herein also include a secondary identifier region located immediately 5′ of the UMI region and having a sequence shared among the primer-adapter nucleic acid molecules. In some embodiments, the secondary identifier region is of between 3 and 10 nucleotides in length (e.g., 3, 4, 5, 6, 7, 8, 9 or 10 nucleotides in length). In some embodiments, the secondary identifier sequence region has a sequence that does not include G nucleotides (e.g., a sequence that only includes A, T and C nucleotides).

In certain embodiments, the primer-adapter nucleic acid molecules described herein are of between 80 and 200 nucleotides in length (e.g., 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 nucleotides in length).

In certain aspects, provided herein is a pair of primer-adapter nucleic acid molecules (or a pair of populations of such molecules) as described herein. In some embodiments, the gene-specific primer region of one of the primer-adapter nucleic acid molecules has a nucleotide sequence that is complementary to the sequence located at the 3′ end of the region of the target nucleic acid to be sequenced, while the gene-specific primer region of the other primer-adapter nucleic acid molecule has a nucleotide sequence that corresponds to the sequence located at the 5′ end of the region of the target nucleic acid to be sequenced.

In some aspects, provided herein is a reaction solution for sequencing a target nucleic acid molecule, the reaction mixture comprising a primer-adapter nucleic acid molecule described herein or a pair of primer-adapter nucleic acid molecules described herein. In some embodiments, the reaction solution further comprises generic primers having the sequence that corresponds to the sequence of generic primer region of a primer-adapter nucleic acid molecule in the reaction solution. In some embodiments, the reaction solution comprises reverse native primers having a shared nucleotide sequence that corresponds to the sequence located at the 5′ end of the region of the target nucleic acid to be sequenced. In some embodiments, the generic primer and/or the reverse native primer is in molar excess compared to the primer-adapter nucleic acid molecules (e.g., at least 2-fold, 3-fold, 4-fold, 5-fold, 6-fold, 7-fold, 8-fold, 9-fold or 10-fold molar excess). In some embodiments, the reaction solution further comprises the target nucleic acid molecule. In some embodiments, the reaction solution further comprises a DNA polymerase (e.g., a thermostable DNA polymerase). In some embodiments, the reaction solution further comprises dNTPs.

In certain aspects, provided herein is a method of generating a sequencing template comprising incubating a reaction solution provided herein under conditions such that the target nucleic acid molecule is amplified to generate a sequencing template. In some embodiments, the reaction solution is incubated under conditions such that the target nucleic acid molecule is amplified for no more than 5 amplification cycles (e.g., for 1, 2, 3, 4 or 5 cycles), the reaction solution is contacted with uracil-DNA-glycosylase to degrade uracil-containing primer-adapters, and then the reaction solution is further incubated under conditions such that the target nucleic acid molecule is further amplified to generate a sequencing template. In some embodiments, the reaction solution is first incubated for no more than 5 cycles (e.g., for 1, 2, 3, 4 or 5 cycles) using an annealing temperature that is less than the melting temperature of the generic primer region of the primer-adapter for its complement, and then further incubated using an annealing temperature that is higher than the melting temperature of the generic primer region but lower than the melting temperature of the generic primer region for its complement. In some embodiments, the amplification process is run for at least 10 cycles in total (e.g., for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cycles). In some embodiments, the sequencing template produced is at least 1,500 bp in length (e.g., at least 2,000 bp, at least 2,500 bp, at least 3,000 bp, at least 3,500 bp, at least 4,000 bp, at least 4,500 bp, at least 5,000 bp, at least 5,500 bp, at least 6,000 bp, at least 6,500 bp, at least 7,000 bp, at least 7,500 bp, at least 8,000 bp, at least 8,500 bp, It least 9,000 bp, at least 9,500 bp, or at least 10,000 bp in length). In some embodiments, the method further comprises sequencing the sequencing template (e.g., using a third-generation sequencing technology, such as single molecule real-time (SMRT) sequencing).

In certain embodiments, the methods provided herein can be used to amplify any target nucleic acid. In some embodiments, the target nucleic acid is a bacterial nucleic acid (e.g., a 16S ribosomal nucleic acid, a drug-resistance gene, a nucleic acid encoding a bacterial antigen). In some embodiments, the target nucleic acid is a viral or retroviral nucleic acid (e.g., a drug-resistance gene, a nucleic acid encoding a viral antigen). In some embodiments, the target nucleic acid is a human nucleic acid (e.g., a cancer-associated gene, such as an oncogene or a tumor suppressor gene). In some embodiments, the region of the target nucleic acid that is sequenced is at least 1,500 bp in length (e.g., at least 2,000 bp, at least 2,500 bp, at least 3,000 bp, at least 3,500 bp, at least 4,000 bp, at least 4,500 bp, at least 5,000 bp, at least 5,500 bp, at least 6,000 bp, at least 6,500 bp, at least 7,000 bp, at least 7,500 bp, at least 8,000 bp, at least 8,500 bp, 1 t least 9,000 bp, at least 9,500 bp, or at least 10,000 bp in length).

### BRIEF DESCRIPTION OF FIGURES

FIG. 1 shows a schematic depiction of an exemplary amplification process according to certain embodiments described herein. Panel A illustrates the structure of primer-adapters and the general composition of reaction mixtures according to certain embodiments provided herein. Panel B illustrates the first amplification cycle and panel C illustrates the second amplification cycle according to certain embodiments of the methods provided herein. Panel D illustrates UDG treatment and the third amplification cycle according to some embodiments of the methods provided herein. Panel E illustrates the fourth and subsequent amplification cycles according to some embodiments of the methods provided herein.

FIG. 2 illustrates the principle of the UMI-driven error correction. Each amplicon off of the original molecule (4 of them are shown on the left-hand side of the figure) is “barcoded” during the first PCR cycle with a unique identifier (UMI) introduced via a UMI adapter primer (as illustrated in FIG. 3). These molecules may contain some true mutations (white stripes), which need to detected. A few PCR-derived errors and a massive number of sequencing errors (dark stripes) are introduced downstream of the barcoding. However, these errors vary from read-to-read, so they can be corrected by making consensus sequences from the sequence reads that share a common UMI label (and are therefore derived from a common original molecule.

FIG. 3 shows an exemplary UMI adapter-primer, the scheme of it's incorporation into the PCR product and the final UMI-containing PCR product according to certain embodiments disclosed herein.

FIG. 4 shows an exemplary sequencing read. The UMIs are underlined and also indicated with capital letters. Of note, these sequences were generated from the opposite strand as the adapter primer, therefore the UMI excludes cytosine, not guanine. Also, the entire primer-adapter is presented here in the reverse complement orientation (hence reversed order: 5′ native primer-GGTTTTTTAAAAGAGA-atgatg (secondary identifier)-spacer-artificial primer 3′). This figure demonstrates the ability to incorporate and read a UMI into a long single molecule. In this case, the molecule containing this UMI was 13 kb long.

FIG. 5 illustrates an exemplary application of an embodiment of the technology disclosed herein. In many important applications it is crucial to distinguish closely related genomes differing by a combination of several nucleotide changes (haplotype) distributed across thousands of base pairs in a complex mixture of closely related genomes, e.g. for microbiome analysis. The conventional short sequence approaches (such as Illumina sequencing) would be unable to distinguish such genomes. This is because when short fragments are analyzed, the linkage between nucleotide changes comprising a haplotype and thus residing on one DNA molecule are lost, and we will not be able to recognize these mutations as a part of a combination. The figure illustrates the fact that with short read sequencing approaches, the output is the same whether sertain combination of nucleotide changes reside on the same molecule or different molecules. In contrast, rare variants can be readily identified by long sequencing using the high fidelity long read single molecule sequencing method according to embodiments described herein. Of note, in addition to mere distinguishing closely related genomes, long fragment sequencing allows for much more efficient de novo sequencing of closely related variants directly from mixtures without the need for sub-cloning (which in many cases is difficult or impossible to perform).

### DETAILED DESCRIPTION

In certain aspects, provided herein are compositions and methods related to the use of unique molecular identifiers (UMIs; i.e. short, randomly generated nucleotide sequences uniquely attached to single DNA molecules) to improve the error-correction capability of third generation sequencing and similar approaches that involve high precision reading of long segments of single DNA molecules. As described herein, these UMIs act as a molecular DNA barcode and allow amplicons generated in an amplification reaction to be traced back to the original target molecule from which they originated. The methods and compositions provided herein therefore can enhance the performance of a sequencing process by increasing the length of continuous DNA fragments that can be sequenced as a single read without sacrificing sequencing fidelity, and can also control for artifact formation during PCR.

In certain embodiments, the methods provided herein include the combination of introducing UMIs into PCR fragments using primer adapters with their subsequent inactivation. In some embodiments, this can serve two purposes: 1) it allows the analysis of very small clinical and/or environmental samples 2) it allows the analysis from a very small number of initial copies, which is critical for long-read sequencing applications such as NanoPore and PacBio sequencing. These “third generation” sequencing applications previously were not suitable for UMI approaches which previously required large copy numbers to be analyzed. Though this requirement is met in certain next generation sequencing methods, such as in Illumina sequencing applications, where hundreds of millions of reads are analyzed, such methods are limited to short reads, which prevents the identification of combinations of variant sequences spread over several kb of sequence. In contrast, third generation long read methods previously were able to be applied to no more than tens of thousands of long reads, and it is unlikely that this number will significantly increase in the future. Thus, for an appropriate representation of UMIs (dozens of copies of each UMI, as needed for the error correction approach), the analysis must be started from no more than thousands of long molecules, which imposes harsh limitations on the PCR procedure. The specific combination of approaches described herein therefore allows the application of the UMI approach to third generation long-read sequencing technologies.

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, two nucleic acid sequences “complement” one another or are “complementary” to one another if they base pair one another at each position.

As used herein, two nucleic acid sequences “correspond” to one another if they are both complementary to the same nucleic acid sequence.

As used herein, the Tm or melting temperature of two oligonucleotides is the temperature at which 50% of the oligonucleotide/targets are bound and 50% of the oligonucleotide target molecules are not bound. Tm values of two oligonucleotides are oligonucleotide concentration dependent and are affected by the concentration of monovalent, divalent cations in a reaction mixture. Tm can be determined empirically or calculated using the nearest neighbor formula, as described in Santa Lucia, J. PNAS (USA) 95:1460-1465 (1998), which is hereby incorporated by reference.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

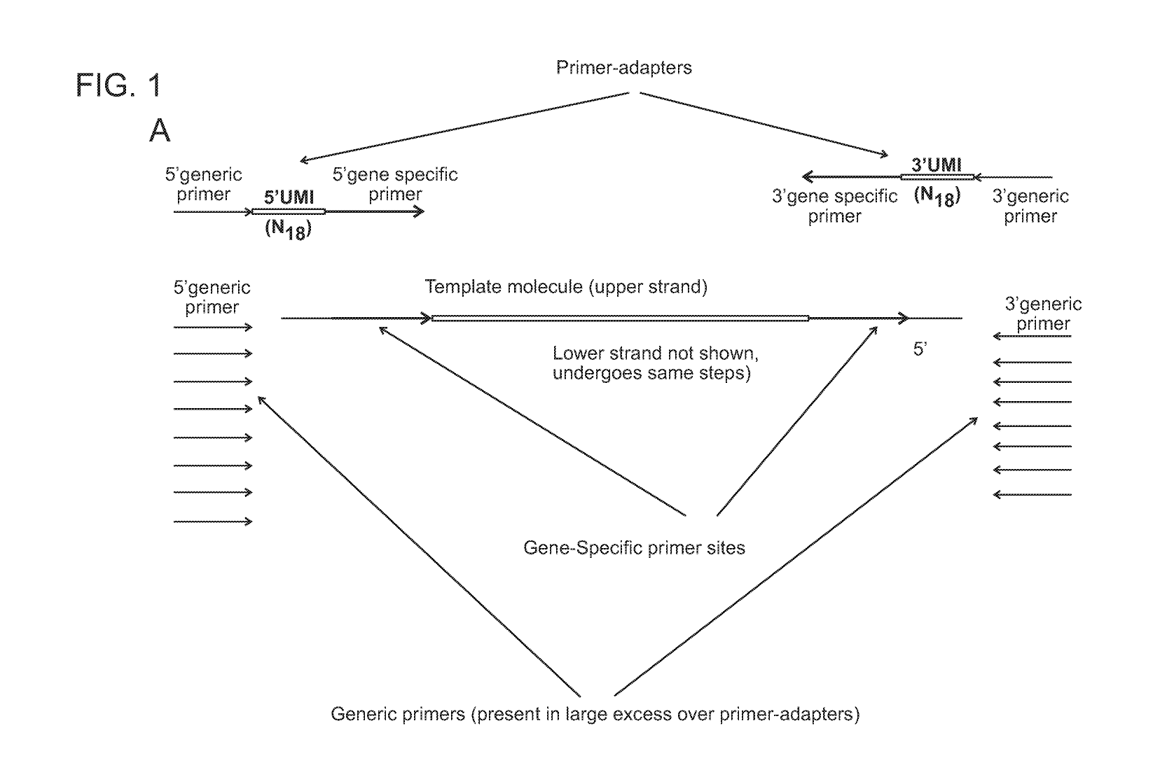

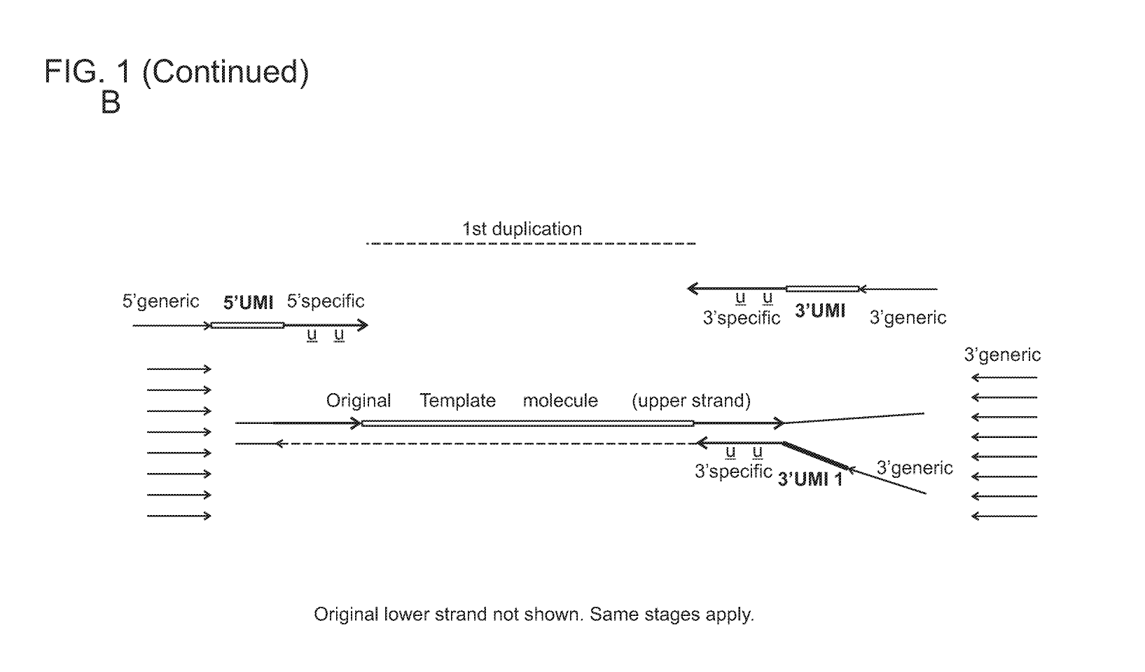

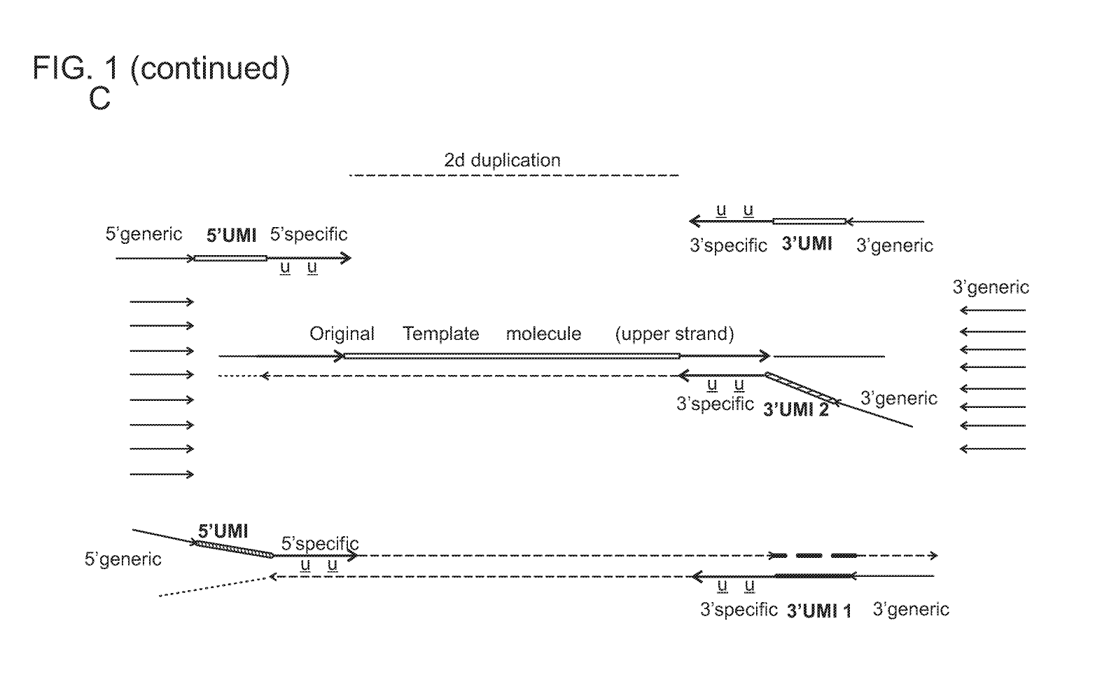

An embodiment of certain methods and compositions described herein is illustrated in FIG. 1. Amplification of an selected genomic locus (up to 15,000 bp) is initiated by the adapter-primers described herein. In some embodiments, two adapter-primers: the 5′ adapter-primer and the 3′ adapter-primer. In some embodiments, an adapter-primer is a synthetic oligonucleotide composed of three functional blocks (or regions): 1) generic primer (3′ or 5′), i.e. an artificial primer sequence; 2) Unique Molecular Identifier (UMI) block, i.e. short random sequence which is synthesized using 4-fold degenerate nucleotide approach (i.e. all four nucleotide precursors are added into reaction at about equimolar concentrations) or 3-fold degenerate nucleotide approach (e.g., A, T and C nucleotide precursors are added into reaction at about equimolar concentrations). As a result, each molecule of the adapter-primer carries a unique combination of nucleotides (e.g., in some embodiments, 18 nucleotides) within this block, which will be attached to the PCR product that generated by amplification using this adapter-primer and all the PCR products derived therefrom; and 3) a locus-specific primer, which is one primer (3′ or 5′) from a regular primer pair designed for specific amplification of a genomic fragment of choice.

In some embodiments, any primer-based amplification process can be used in the methods described herein. Examples of nucleic acid amplification processes include, but are not limited to, polymerase chain reaction (PCR), ligase chain reaction (LCR), strand displacement amplification (SDA), transcription mediated amplification (TMA), self-sustained sequence replication (3SR), and nucleic acid sequence-based amplification (NASBA). In some embodiments, the amplification method is PCR amplification.

In some embodiments, the gene-specific part of the primer-adapter contains uracil in place of thymine. This is done to allow for deactivating of these primers after they complete their task in the 3d duplication cycle by adding UDG (uracil-DNA-glycosilase.

In certain embodiments, the method provided herein allows the labeling of PCR-generated descendants of each original DNA template molecule in the sample with a shared molecular identifier nucleotide sequence, unique for each original template molecule. In some embodiments, this can serve two purposes:

1) Reduction of instrumental error. Each original template will be sequenced multiple times, which allows for the high (˜15%) instrumental error inherent to all third generation sequencing methods to be dramatically reduced, as instrumental error is almost random and thus progressively averages out when increasing number of independent sequences of the same molecule are compared.

2) Reduction of amplification error. Amplification reactions, such as PCR, cause random mutations as amplification proceeds, with the cumulative error level increasing linearly with the number of PCR cycles. Similar to instrumental error, random PCR mutations are averaged out as increasing number of independent sequences of the same molecule are compared. The different variants of the technology described herein have different capacity of correcting PCR errors.

Embodiments provided herein are flexible with respect to the fidelity/complexity trade-off. There are several variants of the technique provided herein.

In certain embodiments provided herein, locus-specific primers in PCR are replaced with the corresponding primer-adapters in combination with generic primers. In the first few cycles of PCR, the primer-adapters initiate DNA synthesis on the targeted nucleic acid and simultaneously attach to the resulting amplicons a UMI and the target site for the generic primer so that in subsequent cycles amplification can be performed using the generic primers. In some embodiments, the melting temperature of the locus-specific primers is lower than the melting temperature of the generic primers. In such embodiments, the first few rounds of amplification can be performed using a primer annealing temperature at which both primers are able to bind, but subsequent rounds can be performed using a primer annealing temperature at which the generic primers can bind but the locus-specific primers cannot. The resulting PCR fragments will carry UMI that is specific to the original template molecules, as required for the proper application of the invention.

In certain embodiments, the primers-adapters comprise several functional blocks collectively allowing the tagging of the PCR progeny of a single molecule template with a specific UMI, as shown in FIG. 1. This variant of the procedure provided herein allows for the reduction of the level of PCR noise in proportion to the number of PCR duplications necessary to amplify the samples, e.g., by at least about 20-fold. In other embodiments, this procedure is used in combination with specific restriction enzyme or CRISPR-Cas9, which are used to cut the cellular DNA next to the adapter-primer binding site. This allows to repeated reads from the same original template molecule to be distinguished by labeling them with different UMIs, which completely excludes PCR noise.

In some embodiments described herein, the primer adapters are inactivated after incorporation of the UMI and generic primer site. This procedure prevents re-priming of the PCR fragments with primer-adapters, which could otherwise rewrite the UMIs and compromise the procedure. In some embodiments the a) UDG based procedure is substituted with the use of b) complementary inhibitory oligonucleotides, c) temperature-dependent suppression of priming and d) restriction endonuclease-dependent deactivation of primer adapters.

In some embodiments, the UMIs are three nucleotide degenerate sequences that lack any G nucleotides. In some embodiments, the use of such UDI sequences enhance long fragment application. PCR reactions of small copy number samples to create large amplicons, which is characteristic of certain applications of the methods provided herein, is sensitive to PCR aberrations, including amplification of parasitic PCR fragments resulting from aberrant priming. Particularly problematic is the formation of “primer dimers”, i.e. short PCR fragments generated from self-priming of the PCR primers. The use of long UMI sequences necessary for third generation sequencing (because of its inherent high instrumental rate that precludes the use of short UMIs) makes this approach highly prone to primer dimer formation and makes long PCR in the presence of conventional UMIs challenging. Using the UMIs provided herein that have three nucleotide degenerate sequences overcomes such issues.

Certain embodiments of the methods and compositions provided herein can be particularly useful for numerous applications, including, for example:

a. In some embodiments, the methods and compositions provided herein are particularly useful for microbiome sequencing. The human microbiome includes a diverse spectrum of microbial organisms that not only coexist within our tissues but also actively participate in a multitude of health and disease states. Additionally, resident microbiomes are unique to specific body regions, organs and tissues, and are individual in nature, meaning that there is extreme diversity between even similar individuals within any given population. Microbiomes can contain thousands of different microorganisms, with diverse growth patterns and profiles and variants. A growing body of evidence strongly supports that alterations in the microbiome are causally related to functions as diverse as digestion and brain function. The extreme heterogeneity and diversity of human microbiomes makes their composition difficult to analyze using current technology. The methods and compositions provided herein enable sequencing of microbiomes with high fidelity, without loss of low abundance or highly similar fractions.

b. In some embodiments, the methods and compositions provided herein also enable sequencing of environmental microbes occurring under natural conditions, which exist in heterogeneous populations, under varying conditions that favor, permit, or prohibit growth.

c. In some embodiments, the methods and compositions provided herein can be applied to sequencing of microbial environmental contaminants.

d. In some embodiments, the methods and compositions provided herein can be used to sequence an infection for the detection of mixed microbial populations or highly similar variants (e.g. mutations resulting in drug resistance)

e. In some embodiments provided herein the high fidelity sequencing enabled by the methods and compositions provided herein will allow the detection of rare sequences, and an application of this aspect would be to determine if a compound is mutagenic (i.e. toxicology screening).

f. In some embodiments, the methods and compositions provided herein can be used as a partial diagnostic for oncogenic somatic mutation screening.

### EXAMPLE

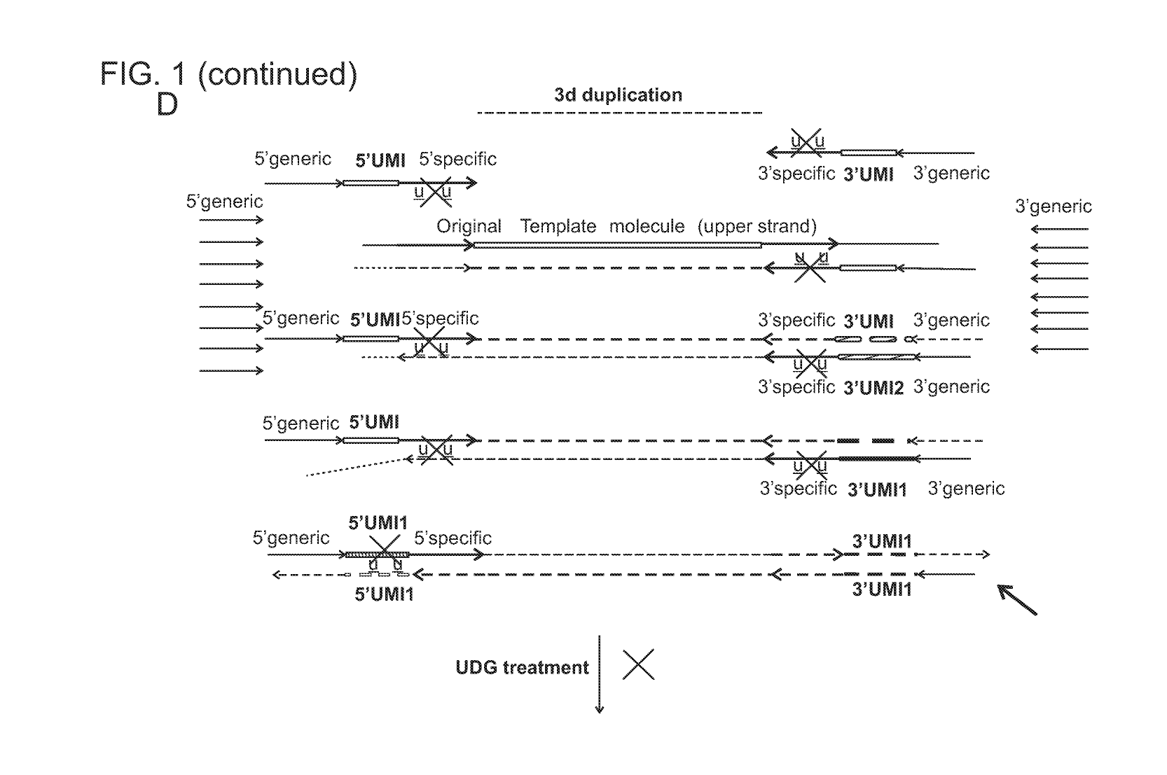

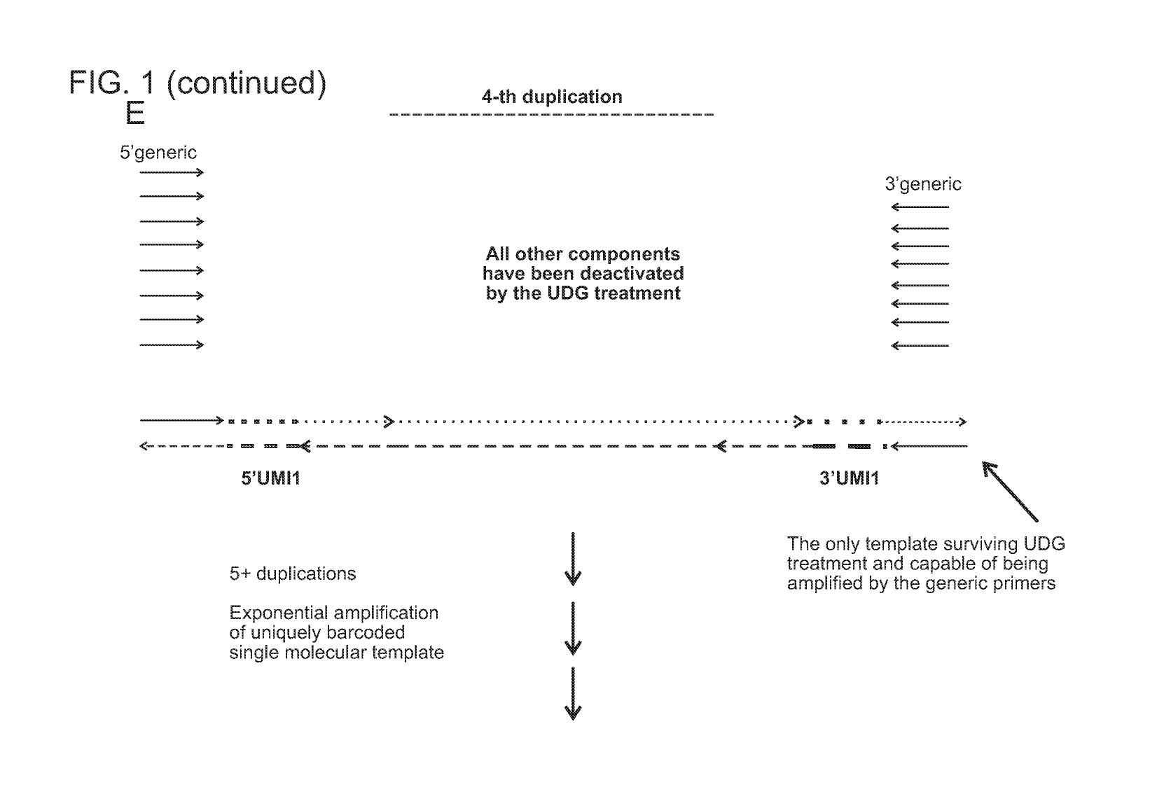

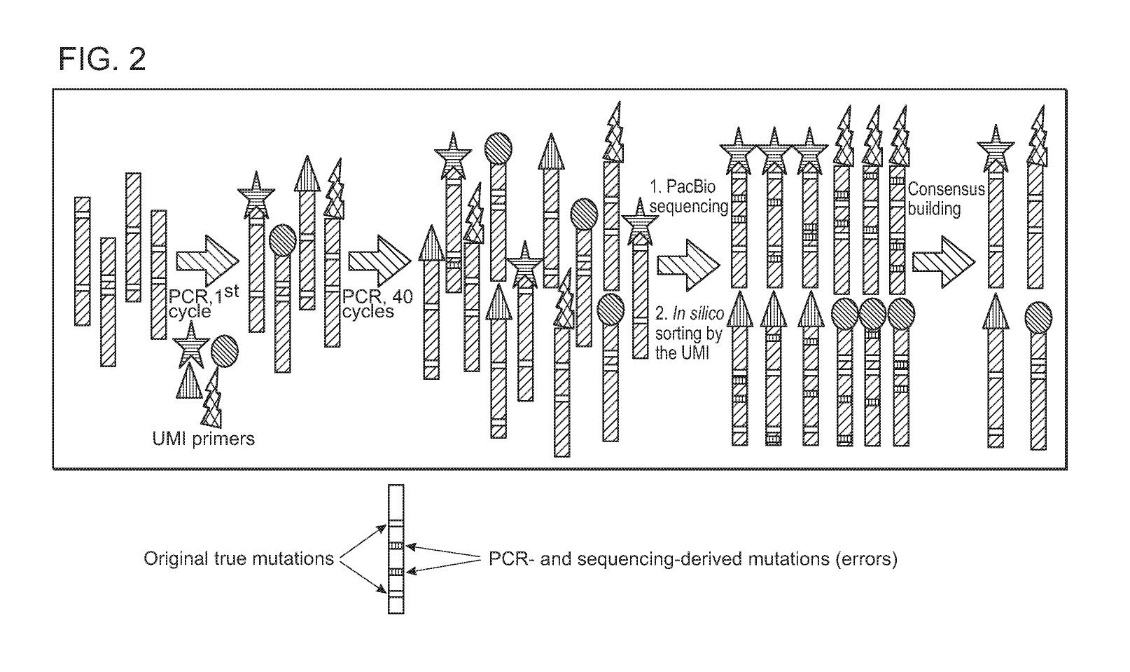

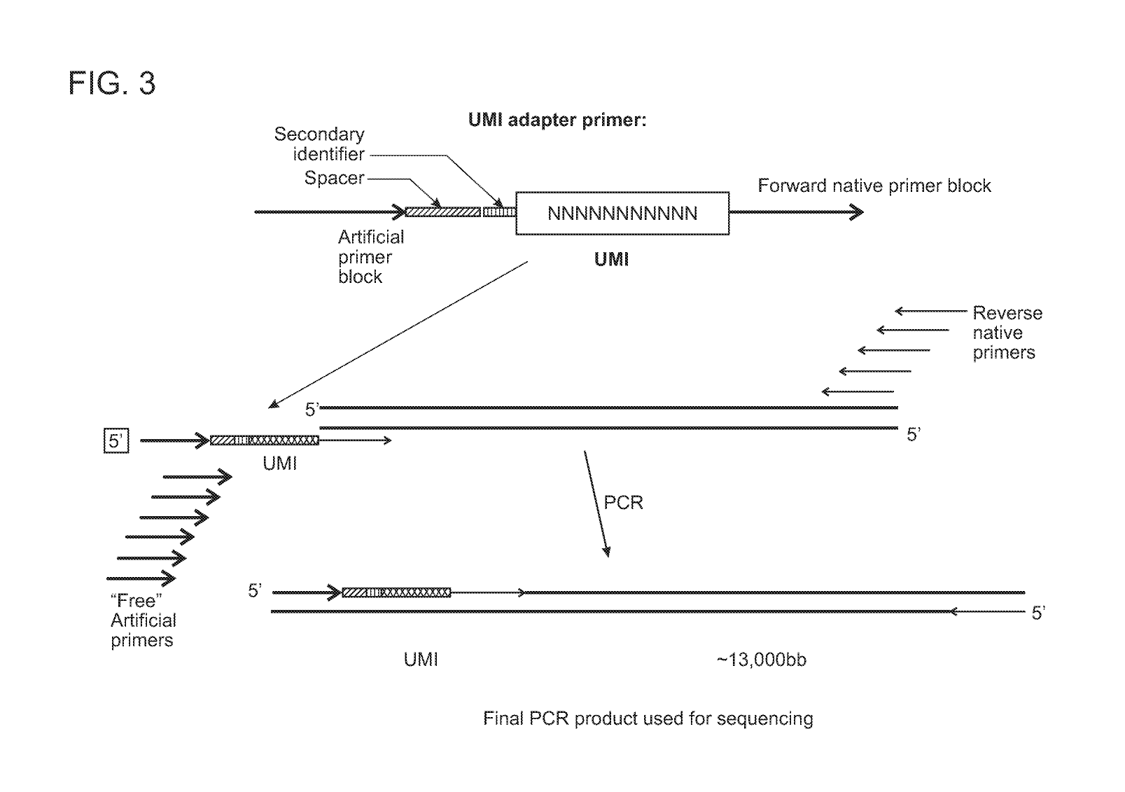

A sequencing of about 100 individual molecules, each about 13,000 bp long and barcoded with unique molecular identifiers (UMIs) was performed in a single PacBio sequencing run. FIG. 2 illustrates the principle of the UMI-driven error correction used. Every original molecule (4 of them are shown the left) is “barcoded” during the first PCR cycle with a unique identifier (UMI) introduced via a UMI adapter primer (as shown in detail in FIG. 3). These molecules may contain some mutations (white stripes) which will be revealed by sequencing. A few PCR-derived errors and a massive number of sequencing errors (dark stripes) are introduced downstream of the barcoding. Importantly, these errors are different in different reads, so they can be corrected by building a consensus sequence.

The object of the methods provided herein is to produce long reads from individual molecules with high fidelity. Using the method described herein, in this example, single molecule reads up to ˜12,000 bp long (average 7,300 bp) with no substitution errors per 110,000 base pairs recovered so far (excluding indels), that is, 99.999+% accuracy, or Phred score of 50+ have been achieved. This is a more than 4-orders of magnitude improvement over the innate PacBio sequencing error rate of about 15-20%. While the methods provided herein can allow some indel (deletion) errors, these are limited to rare special sequences (for example, long polyA tracts (e.g., A₁₁) that are known as highly problematic for PacBio sequencing. These latter type of errors is easily identified as such and the corresponding sites, if they happen to be present, can be excluded from analysis.

The UMI adapter primer used was a 125 base pair (bp) single-stranded oligonucleotide synthesized by Eurofin genomics. The 3′ region of the adapter is complementary to a specific 28 bp region found in the mouse mitochondrial genome and can be used as a PCR primer (Forward Native primer block). For additional applications, this region can be altered to create complementarity to any desired species or gene target.

Adjacent to the native primer region on the adapter is the random UMI, consisting of a long (16 bp+), unknown random sequence of A, T, and C, synthesized using degenerate synthesis with three nucleotide precursors added at every step. Guanine bases were excluded to reduce the amount of random homology between the UMI and the DNA template. Upstream of the UMI is a secondary identifier, a ˜6 bp sequence that can be altered on different adapter constructs to allow for the pooling of diverse samples on a single sequencing chip (this secondary identifier is analogous to “index” sequence in Illumina). The remaining 5′ region was an arbitrary selected sequence of A, T, and C created as a space buffer (“spacer”). This “spacer” is useful to ensure the readability of the UMI because in a typical PacBio sequencing read, the initial ˜60 bp are poor quality and unusable, so. The 5′ region is used as a priming site in PCR so that the template DNA can be amplified along with its attached UMI (“artificial primer”).

The UMI adapter was attached to the target DNA molecule during the first cycle of a PCR reaction. TaKaRa LA Taq hot-start DNA polymerase was chosen for the PCR due to its ability to robustly amplify long templates at low copy number. The reaction contained three primers: a reverse primer native to the target template at 0.2 the forward adapter primer at 0.02 and a forward primer starting at the 5′ end of the adapter at 0.2 μM. The reduced concentration of the adapter primer significantly lowers the chances of that primer to anneal to the template DNA. This effectively prevents a single template molecule from being primed and therefore re-identified multiple times. Once some DNA templates have the adapter incorporated into them, the full-concentration primer at the 5′ end of the adapter will work to efficiently amplify the adapter/DNA construct. The cycling conditions start with a 30 second denaturing step at 95 degrees, followed by 45 cycles of a 30 second, 90 degree denaturing step and a 16 minute, 68 degree combined annealing in extension step, with a 6 minute 68 degree final extension.

As the UMI is a random sequence of nucleotides, there is a high probability for the primers in the PCR to anneal and amplify the primer adapter itself, although this effect was greatly diminished by removing guanines from that region. In order to purify the sample for sequencing, the Monarch Gel Extraction Kit from New England Biolabs was used to specifically cut the target band out of the gel, leaving behind the smaller non-specific fragments. If a greater quantity of PCR product is needed, the extracted sample can be amplified again in a new reaction without the adapter primer present. This will lead to a greater quantity of UMI-labeled product without the interference of non-specific short fragments.

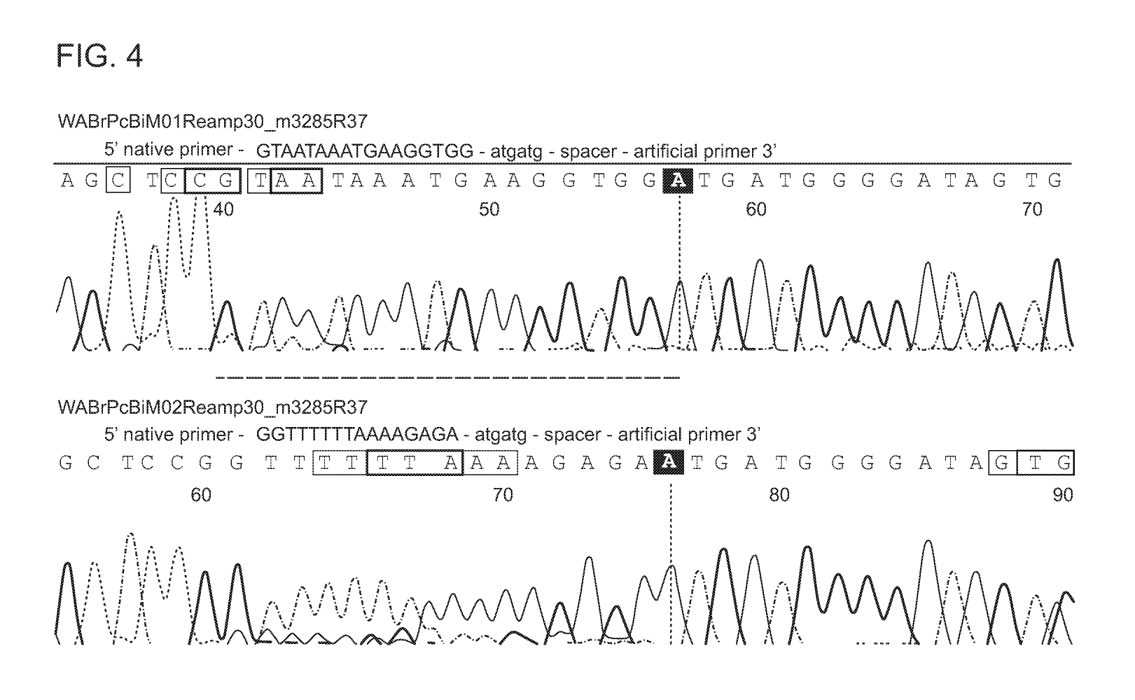

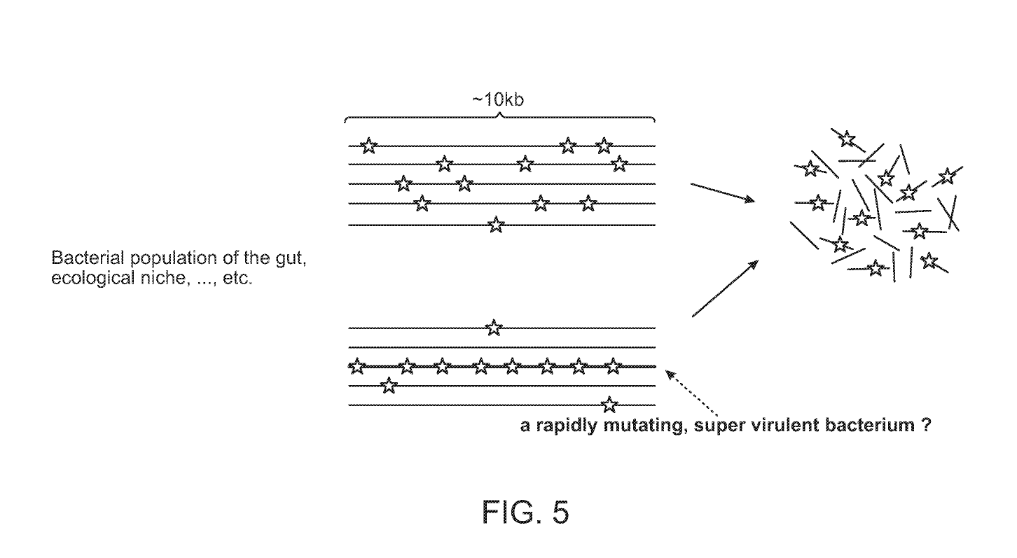

In order to test that this methodology was able to attach single UMIs to a target DNA sequence, initial experiments were conducted at the single DNA molecule level and verified with Sanger sequencing. DNA samples were diluted to very low copy number and amplified such that each positive well represented amplification from a single starting template. Sanger sequencing revealed that single, clear UMIs were attached to the target sequence as expected. Exemplary sequencing results are shown in FIG. 4.

After initial Sanger sequencing, the library was sent PacBio sequenced, and the data parsed using a data pipeline that locates and extracts UMI sequence from each PacBio read and further performs clustering of the UMI dataset, while maintaining connection of the UMIs to the parent reads. Sequence reads corresponding to the UMI clusters were called and their consensuses generated using PacBio LAA long read consensus builder. Because reads within each cluster bared the same UMI, they were derived from the same original molecule and therefore their consensus represented the sequence of that original molecule.

The sequence analysis demonstrated that each mtDNA molecule had been marked, in addition to the artificially attached UMI, by a constellation of innate random mutations, which independently uniquely identified each molecule. It was therefore confirmed that each read UMI-defined cluster of reads indeed represented a single original molecule. Indeed in all 10 clusters analyzed consisted exclusively of the reads that traced back to a single molecule.

To determine the accuracy of the approach, a 50%-jackknife-type super-consensus reconstruction procedure was performed. Each cluster of reads was randomly split into two parts four times, resulting in 8 different, random-sampled, equally sized sub-clusters. An LAA consensus was constructed from each sub-cluster using PacBio software. The consensus sequences were aligned using MAFFT and trimmed to the shortest one in the alignment and further to remove any low quality alignment ends. Then super-consensus was constructed following the 100% rule. A position in a sequence was considered “reliable” only if all 8 resulting consensuses agreed with respect to the nucleotide in that position. Of 110,000 reads, there were 3 unreliable positions, 2 included both deletions and a non-reference nucleotide, and one included 2 consensuses with a discordant nucleotide. There were a couple dozen unreliable sites with deletions. The 100% deletions were limited to the special sequence, A₁₂.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

1. A population of 5′ primer-adapter nucleic acid molecules for sequencing a region of a target nucleic acid, each primer-adapter nucleic acid molecule in the population comprising, in 5′ to 3′ order:
(a) a 5′ generic primer region having a nucleotide sequence shared among the members of the population of 5′ primer-adapter nucleic acid molecules and that is not complementary to a sequence of the target nucleic acid;
(b) a 5′ unique molecular identifier (UMI) region having a sequence that differs between each member of the population of 5′ primer-adapter nucleic acid molecules; and
(c) a 5′ gene-specific primer region having a nucleotide sequence shared among the members of the population of 5′ primer-adapters nucleic acid molecules and that is complementary to the sequence located at the 3′ end of the region of the target nucleic acid to be sequenced.

2. (canceled)

3. The population of 5′ primer-adapter nucleic acid molecules of claim 1, further comprising a 5′ spacer region of between 10 and 100 nucleotides in length positioned between the 5′ generic primer region and the 5′ UMI region.

4. The population of 5′ primer-adapter nucleic acid molecules of claim 3, wherein the 5′ spacer sequence region has a sequence consisting of A, T and C nucleotides.

5. The population of 5′ primer-adapter nucleic acid molecules of claim 3, further comprising a 5′ secondary identifier region of between 3 and 10 nucleotides in length positioned between the spacer region and the 5′ UMI region and having a sequence shared among the members of the population of 5′ primer-adapter nucleic acid molecules.

6-10. (canceled)

11. The population of 5′ primer-adapter nucleic acid molecules of claim 1, wherein the 5′ gene-specific primer region comprises one or more U nucleotides.

12. The population of 5′ primer-adapter nucleic acid molecules of claim 11, wherein the 5′ gene-specific primer region comprises U nucleotides in place of T nucleotides.

13. (canceled)

14. The population of 5′ primer-adapter nucleic acid molecules of claim 1, wherein the target nucleic acid is a bacterial nucleic acid.

15-16. (canceled)

17. The population of 5′ primer-adapter nucleic acid molecules of claim 1, wherein the target nucleic acid is a viral nucleic acid.

18. The population of 5′ primer-adapter nucleic acid molecules of claim 1, wherein the target nucleic acid is a human nucleic acid.

19. The population of 5′ primer-adapter nucleic acid molecules of claim 1 wherein the target nucleic acid is a cancer-associated gene.

20. The population of 5′ primer-adapter nucleic acid molecules of claim 19, wherein the cancer-associated gene is an oncogene or a tumor suppressor gene.

21. A pair of populations of primer-adapter nucleic acid molecules for sequencing a target nucleic acid, the pair of populations comprising:
(a) the population of 5′ primer-adapter nucleic acid molecules of claim 1; and
(b) a population of 3′ primer-adapter nucleic acid molecules, each primer-adapter nucleic acid molecule in the population comprising, in 5′ to 3′ order:
(i) a 3′ generic primer region having a nucleotide sequence shared among the members of the population of 3′ primer-adapter nucleic acid molecules and that is not complementary to a sequence of the target nucleic acid;
(ii) a 3′ unique molecular identifier (UMI) region having a sequence that differs between each member of the population of 3′ primer-adapter nucleic acid molecules; and
(iii) a 3′ gene-specific primer region having a nucleotide sequence shared among the members of the population of 3′ primer-adapters nucleic acid molecules and that corresponds to the sequence located at the 5′ end of the region of the target nucleic acid to be sequenced.

22-40. (canceled)

41. A reaction solution for sequencing a target nucleic acid molecule, the reaction solution comprising:
(a) the pair of populations of primer-adapter nucleic acid molecules of claim 21;
(b) a population of 5′ generic primers, having the sequence of the 5′ generic primer region of the population of 5′ primer-adapter nucleic acid molecules; and
(c) a population of 3′ generic primers, having the sequence of the 3′ generic primer region of the population of 3′ primer-adapter nucleic acid molecules.

42-45. (canceled)

46. The reaction solution of claim 41, further comprising the target nucleic acid molecule.

47. The reaction solution of claim 46, further comprising a DNA polymerase and dNTPs.

48. A reaction solution for sequencing a target nucleic acid molecule, the reaction solution comprising:
a) the population of 5′ primer-adapter nucleic acid molecules of claim 1;
(b) a population of 5′ generic primers, having the sequence of the 5′ generic primer region of the population of 5′ primer-adapter nucleic acid molecules; and
(c) a population of 3′ reverse native primers, having a shared nucleotide sequence that corresponds to the sequence of a region of the target nucleic acid located at the 5′ end of the region of the target nucleic acid to be sequenced,

49-50. (canceled)

51. The reaction solution of claim 48, further comprising the target nucleic acid molecule.

52. The reaction solution of claim 51, further comprising a DNA polymerase and dNTPs.

53. A method of generating a sequencing template comprising incubating the reaction solution of claim 47 under conditions such that the target nucleic acid molecule is amplified to generate a sequencing template.

54-55. (canceled)

56. A method of generating a sequencing template comprising the steps of:
(a) incubating the reaction solution of claim 47 under conditions such that the target nucleic acid molecule is amplified for less than 5 amplification cycles;
(b) contacting the reaction solution with uracil-DNA-glycosylase to degrade uracil-containing primer-adapters; and
(c) incubating the reaction solution under conditions such that the target nucleic acid molecule is further amplified to generate a sequencing template.

57-61. (canceled)


SEQUENCE LISTING (4 sequences)

SEQ ID NO: 1 - DNA, 22 bp, Artificial Sequence
  Description of Artificial Sequence Synthetic oligonucleotide
ggttttttaaaagagaatgatg

SEQ ID NO: 2 - DNA, 23 bp, Artificial Sequence
  Description of Artificial Sequence Synthetic oligonucleotide
gtaataaatgaaggtggatgatg

SEQ ID NO: 3 - DNA, 38 bp, Artificial Sequence
  Description of Artificial Sequence Synthetic oligonucleotide
agctccgtaataaatgaaggtggatgatggggatagtg

SEQ ID NO: 4 - DNA, 36 bp, Artificial Sequence
  Description of Artificial Sequence Synthetic oligonucleotide
gctccggttttttaaaagagaatgatggggatagtg